(19) United States
(12) Patent Application Publication
(10) Pub. No.: US 2010/0268254 A1
(43) Pub. Date: Oct. 21, 2010

(54) Clamp System and Method of Using the Same

(76) Inventors: Joe Alan Golden, San Antonio, TX (US); Alfred H. Raschdorf, JR., Kings Park, NY (US)

Correspondence Address: GUNN, LEE & CAVE, P.C., 300 CONVENT ST., SUITE 1080, SAN ANTONIO, TX, 78205, US

(21) Appl. No.: 12/740,931

(22) PCT Filed: Nov. 12, 2008
(86) PCT No.: PCT/US2008/083269

§ 371 Date: Apr. 30, 2010

Related U.S. Application Data
(60) Provisional application No. 60/987,372, filed on Nov. 12, 2007.

(51) Int. Cl. A61B 17/08; A61B 17/10

(52) U.S. Cl. 606/142; 606/157

(57) ABSTRACT

A system for deploying clamps and methods of using the same are disclosed. The system can be configured to be inserted through a percutaneous trocar with a releasably attached clamp in a closed configuration. The system can be configured to then open the clamp and position the clamp around a target vessel. The system can be configured to then close the clamp over the vessel, squeezing the vessel to partially or completely close an internal lumen in the vessel. The clamp can be detached from the remainder of the deployment system and left on the vessel, or the clamp can be opened and removed.

### CROSS-REFERENCE TO RELATED APPLICATION

This application claims priority to U.S. Provisional Application No. 60/987,372, filed 12 Nov. 2007, which is incorporated by reference herein in its entirety

### BACKGROUND OF THE INVENTION

1. Field of the Invention

This invention relates to clamps, clamp deployment systems and methods of using the same. The clamps can be used in biological environments and can be used to clamp biological lumens.

2. Description of Related Art

“Bulldog” clamps are surgical clamps for occluding numerous kinds of bodily vessels and tubular organs, for example, blood vessels, bowel, ducts, urethra, and the like. These clamps can also be used for clamping other anatomical structure such as the lung, liver or adnexa, where it is often necessary to clamp not only for occlusion, but also for retraction. The occlusion of these anatomical structures, especially tubular structures, is often necessary during surgery to prevent leakage of lumen contents at the surgical site.

Bulldog clamps are often designed for temporary clamping. The clamps are often intended to clamp a structure, e.g. occlude a vessel or tubular organ, while the surgery is being performed, and then be subsequently removed from the occluded vessel or organ when the surgery is completed.

In order to facilitate the ability to drop off the bulldog clamp after it is clamped about the vessel or tubular organ, and to thereafter grasp and remove the clamp from the vessel or organ following the surgical procedure, the clamp must possess some kind of configuration in addition to its clamping jaws. Typically, the clamp includes a spring loaded handle mechanism for grasping the clamp and for providing the opposing jaw clamping force. More specifically, it has a self-contained spring element in its handle for providing the constricting force to the desired vessel or tubular organ.

While the current conventional design for bulldog clamps has worked well for surgeons in open surgery, modifications to the existing clamps have been necessary for their adaptation to endoscopic or other minimally invasive surgeries: surgery performed through small ports or openings in the body with the aid of special equipment and surgical instrumentation, typically allowing the surgeon to perform the surgery while observing his operative technique on a video monitor. The small openings are typically made using a cannula (e.g., trocar), a puncturing instrument for providing access through the body wall to the surgical site.

In open surgery, ratcheting scissor-handled type clamps have often been used rather than bulldog clamps. These scissor-handled clamps use the surgeon's hand force and a ratchet mechanism to apply and retain the clamping force. Such clamps cannot fit conveniently down trocars. Ratcheting ring-handled clamps, especially designed for endoscopic use, were developed. These clamps require full-time dedicated trocar ports, thus limiting the number of other instruments which the surgeon can introduce at any one time when the number of trocar ports remain constant.

Since drop-off bulldog clamps provide a tremendous advantage over conventional, scissor-handled clamps during endosurgery, a challenge has been how to introduce these clamps into the body, clamp them onto the desired anatomical structures and in the desired orientation, free the access port for other uses, and then retrieve them through a port when their function is fulfilled. Therefore, it has become necessary to develop a suitable endoscopic applier to apply such drop-off bulldog clamps.

Therefore, a deployment clamp system that can deploy a clamp such as a bulldog clamp is desired that can be deployed through a radially small and/or tortuous, and/or long cannula or trocar. Furthermore, an easily releasable and reattachable clamp and accompanying deployment tool is desired. For example, a deployment clamp system is desired that provides feedback to the user at various stages in the deployment of the clamp.

### SUMMARY OF THE INVENTION

A clamp deployment system for use with biological lumens is disclosed. The clamp deployment system can have a deployment tool releasably attached to a clamp. The clamp can have a first jaw and a second jaw. The jaws can have jaw faces. The jaw faces can have a high-friction surface, such as a knurled surface. The clamp can have open and closed configurations. The open configuration can have the jaws in a radially expanded configuration. The closed configuration can have the jaws in a radially contracted configuration. The jaws can be a radially opening clip and/or a parallel jaw clip.

The deployment system can have a rigid or flexible neck. The neck can rotate or bend.

The deployment tool can have a control element, for example squeezable handles. The control element can control whether the clamp is in an open or closed configuration. The control element can cause the clamp to distally extend away from the neck. The control element can control the release of the clamp from the deployment tool. The control element can provide feedback to the user, for example to notify the user when the clamp is completely closed and before the clamp is extended away from the neck.

The clamp can transform into an open configuration when the clamp is forcibly compressed against the neck. The clamp can transform into a closed configuration when the clamp is in a longitudinally relaxed configuration, for example not forcibly compressed against the neck.

The clamp deployment system can have and/or be used with a cannula, such as a trocar. The cannula can be introduced across a tissue surface, such as percutaneous (across the skin), across the peritoneal membrane, or a specific organ membrane such as the pericardium or bronchial membrane. The clamp can then be deployed through the cannula. The neck can be long enough and flexible enough to extend through the cannula and to conform to tortuous paths defined by the cannula.

The transverse cross-section of the clamp in the closed configuration can be substantially circular. The clamp can be configured. (e.g., with a circular transverse cross-section) to be efficiently deployed through the cannula or trocar. For example, the clamp outer diameter can be 4 mm (0.16 in.) or less.

The deployment tool can be configured to re-attach to the clamp after releasing the clamp.

The clamp can be in a closed configuration and attached to the deployment tool before being moved through the cannula from outside the subject's body to inside the subject's body or inside, and/or through any other tissue surface or membrane. The clamp can be positioned near a target lumen. The clamp can then be opened and positioned around a target vessel having the target lumen. The clamp can then be partially or completely closed on and/or around the target vessel, for example, partially or completely obstructing the flow through the lumen. The clamp can then be extended from the deployment tool and detached from the deployment tool. The deployment tool and/or the cannula can then be removed from the subject.

After the clamp is detached from the deployment tool, the clamp can be re-attached to the deployment tool. Once re-attached to the deployment tool, the clamp can be opened, repositioned over the target vessel or another vessel, and re-closed. Once re-attached to the deployment tool, the clamp can be opened, moved away from the target vessel, closed (e.g., not on a vessel), and withdrawn through the cannula or otherwise.

### BRIEF DESCRIPTION OF THE DRAWINGS

FIG. 1a illustrates a side view of a variation of the clamp deployment system in a configuration with the clamp open.

FIG. 1b illustrates a side perspective view of the variation of the clamp deployment system of FIG. 1a.

FIG. 1c illustrates a from view of the variation of the clamp deployment system of FIG. 1a

FIG. 2 illustrates the variation of the clamp deployment system of FIG. 1a in a configuration with the clamp closed.

FIG. 3 illustrates the variation of the clamp deployment system of FIG. 1a in a configuration with the clamp extended away from the neck.

FIGS. 4a and 4b illustrate variations of the clamp deployment system of FIG. 1a with a return spring.

FIGS. 5a and 5b illustrate a variation of the clamp deployment system in a first and second configuration, respectively.

FIGS. 6a and 6b illustrate a variation of the clamp deployment system in a first and second configuration, respectively.

FIG. 7 is a side view of a variation of the clamp.

FIG. 8a is a top-side view of a variation of the clamp in an open configuration.

FIG. 8b is a top view of the variation of the clamp of FIG. 5a.

FIG. 8c is a rear-side view of the variation of the clamp of FIG. 5a.

FIG. 8d is an exploded view of the variation of the clamp of FIG. 5a.

FIG. 9a illustrates a variation of the clamp in a contracted closed configuration.

FIG. 9b illustrates the clamp of FIG. 9a transforming into an expanded, open configuration.

FIG. 10 is a top view of the clamp deployment system of FIGS. 1a-c.

FIG. 11 illustrates a variation of cross-section A-A.

FIG. 12 illustrates a dose-up cross-section of the clamp and the end of deployment tool of FIG. 11.

FIG. 13 illustrates a close-up cross-section of the clamp and the end of deployment tool of FIG. 12.

FIG. 14 is an exploded view of a variation of the clamp deployment system.

FIG. 15 is a close up of the proximal end of the clamp deployment system of FIG. 14.

FIG. 16 is a top view of the clamp deployment system of FIGS. 1a-c with the clamp in a closed configuration and the deployment tool in a configuration extending the clamp away from the deployment tool.

FIG. 17 illustrates a variation of cross-section B-B.

FIG. 18 illustrates a close-up cross-section of the clamp and the end of deployment tool of FIG. 17.

FIG. 19 illustrates a variation of the clamp deployment system with the deployment tool in a configuration extending the clamp away from the deployment tool.

FIG. 20 illustrates a variation of close-up section C-C.

FIG. 21 illustrates a variation of the clamp deployment system with the deployment tool detached from the clamp.

FIG. 22 illustrates a variation of close-up section D-D.

FIG. 23a illustrates a variation of a clamp deployment system

FIG. 23b illustrates a method of detaching the clamp from the deployment tool of FIG. 23a.

FIGS. 24 through 30 illustrate a variation of a method of using a variation of the clamp deployment system.

### DETAILED DESCRIPTION

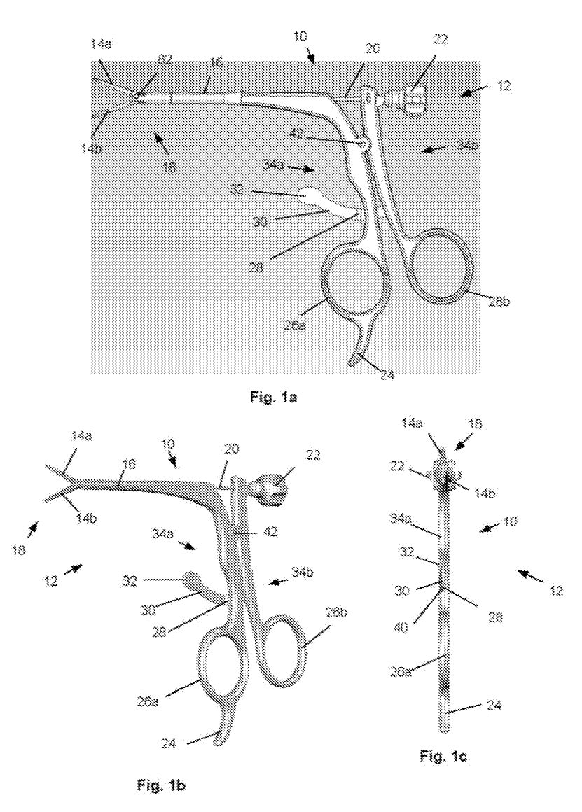

FIGS. 1a, 1b and 1c illustrate a clamp deployment system 12 that can have a clamp 18 fixedly or releasably attached to a deployment tool 10. The clamp 18 can have a first configuration, such as an open and/or radially expanded configuration as shown. The clamp 18 can have a first jaw 14a, a second jaw 14b and a clamp hinge 82. The first jaw 14a can be rotatably attached to the second jaw 14b at the clamp hinge 82. The clip can be a radially opening clip, as shown, or a parallel jaw clip.

The deployment tool 10 can have a neck 16. The neck 16 can be flexible and/or rigid. The neck 16 can be configured to abut the clamp 18 The neck 16 can be resilient and/or deformable. The neck 16 can have a length appropriate for use in various procedures. For example, the neck 16 can have a length from about 5 cm (2 in.) to about 50 cm (20 in.), for example about 32 cm (13 in.).

The deployment tool 10 can have a deployment rod 20. The deployment tool 10 can controllably translate the deployment rod 20, for example along a longitudinal axis of the deployment rod 20. The deployment rod 20 can be rigid and/or flexible. The deployment rod 20 and/or neck 16 can be configured to conform to tortuous configurations. The deployment rod 20 can be removably attached to the clamp 18. The deployment rod 20 can be fixed to a movable handle, for example by a deployment rod anchor 22. The deployment rod anchor 22 can be fixed to the deployment rod 20 and the second handle 34b. The clamp 18, deployment rod 20 and deployment rod anchor 22 can have central longitudinal channels passing therethrough, for example allowing the deployment of one or more guidewires, catheters or other elongated tools through the deployment rod 20 and out the distal end of the clamp 18. The deployment rod anchor 22 can be fixedly or rotatably attached to the deployment rod 20. The deployment rod anchor 22 can be threadably or otherwise adjustably attached to the second handle 34b. For example, the deployment rod anchor 22 can be rotated relative to the second handle 34b to increase or decrease the torque exerted by the clamp 18 (e.g., between the jaws 14) for a given angle between the first handle 34a 34a and the second handle 34b.

The deployment tool 10 can have a first handle 34a 34a and a second handle 34b. The second handle 34b can be rotatably attached to the first handle 34a 34a at a handle hinge 42, The second handle 34b, for example near a first end, can be attached to the deployment rod 20. The first handle 34a 34a and/or the second handle 34b can have one or more loops for inserting a finger or thumb for control of the handle. For example, the first handle 34a 34a can have a first digit loop 26a. The second handle 34b can have a second digit loop 26b. The handles 34 can have an extending digit rest 24, for example for resting a digit and/or controlling the handle. The handles 34 can have additional configurations for improved ergonomics, for example, contours to place additional digits during use.

A handle arm 30 can extend from the second handle 34b. The handle arm 30 can substantially cross adjacent to the first handle 34a 34a. The handle arm 30 can have a stop 32, for example to create an interference fit when the second handle 34b is at the maximum desired rotation with respect to the first handle 34a 34a.

The handle arm 30 can have a marker 28. The marker 28 can be configured to provide feedback when the second handle 34b reaches a selected rotation with respect to the first handle 34a. For example, the marker 28 can vibrate, click, provide a temporary stop against the first handle 34a, or combinations thereof, when the second handle 34b reaches a rotation sufficient to begin extension of the clamp 18 away from the neck 16 (see below).

The stop 32 can also be used as a control surface. For example, a user can press the stop 32 in a direction perpendicular to the flat surface of the stop 32 with the user's thumb. The handle arm 30 can be rotatably attached to the second handle 34b, and/or the handle arm 30 can be resiliently and/or deformably flexible. The pressing can be in a direction that can bend the handle arm 30 away from the first handle 34a. Bending the handle arm 30 out of the way can allow the marker 28 to pass by the first handle 34a during use (e.g., relative rotation of the first handle 34a relative to the second handle 34b) without substantially contacting the first handle 34a, and therefore not having any substantial interference fit against the first handle 34a.

The first handle 34a can have an arm notch 40. The arm notch 40 can be configured to be sized to allow the majority of the handle arm 30 to slide through the arm notch 40 without interference, but not the marker 28 or the stop 32. The arm notch 40 can be configured to have a temporary interference fit with the marker 28. The arm stop can be configured to have a substantially impassable interference fit with the stop 32.

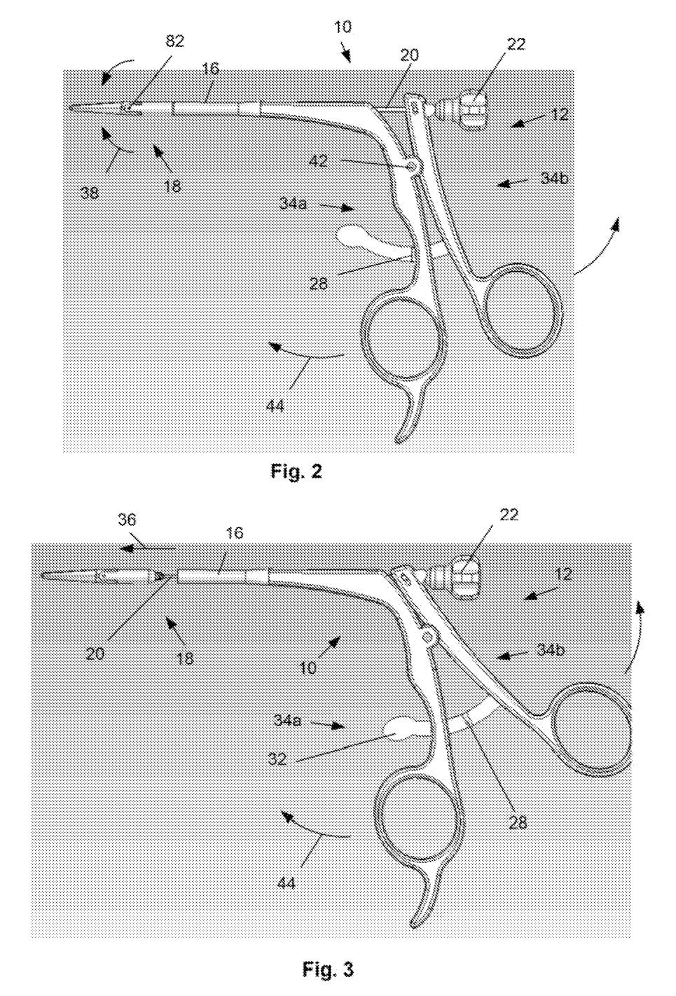

FIG. 2 illustrates that clamp 18 can have a second configuration, such as a closed and/or radially contracted configuration. The clamp deployment system 12 can be transformed from the configuration shown in FIGS. 1a, 1b and 1c to the configuration shown in FIG. 2 by rotating the handles 34, as shown by arrows 44. The rotation of the second handle 34b with respect to the first handle 34a can push (e.g., translate) the deployment rod 20 in the direction of the clamp 18. The deployment rod translation can cause the jaws 14 of the clamp 18 to rotate towards each other (i.e., for the clamp 18 to radially contract), as shown by arrows 38, resulting in a decreased radius of the clamp 18.

With the clamp 18 in the closed configuration, the face of the first jaw 14a can contact or be in close proximity to the face of the second jaw 14b. The marker 28 can abut the first handle 34a, for example creating force feedback via a temporary interference fit (e.g., an interference fit that can be overcome by the application of additional force, reasonably delivered by the user).

The clamp 18 can have a transverse cross-section substantially perpendicular to the longitudinal axis of the clamp 18 and the distal end of the neck 16. In the closed configuration, the transverse cross-section of the clamp 18 can be substantially oval or circular along the length of the clamp 18 (e.g., along substantially the entire length of the closed jaws 14 and the clamp case 64).

FIG. 3 illustrates the clamp deployment system 12 can have a configuration that can have the clamp 18 extended away from the neck 16. The second handle 34b can be rotated away from the first handle 34a, as shown by arrows 44. The clamp 18 can extend (e.g., translate), as shown by arrow, away from the neck 16. The clamp 18 can extend away from the neck 16 due to the relative rotation of the first and second handle 34bs, for example from a range of rotation between a first angle at which marker 28 is pushed beyond the temporary interference fit against the first handle 34a, and a second angle where the stop 32 contacts the first handle 34a.

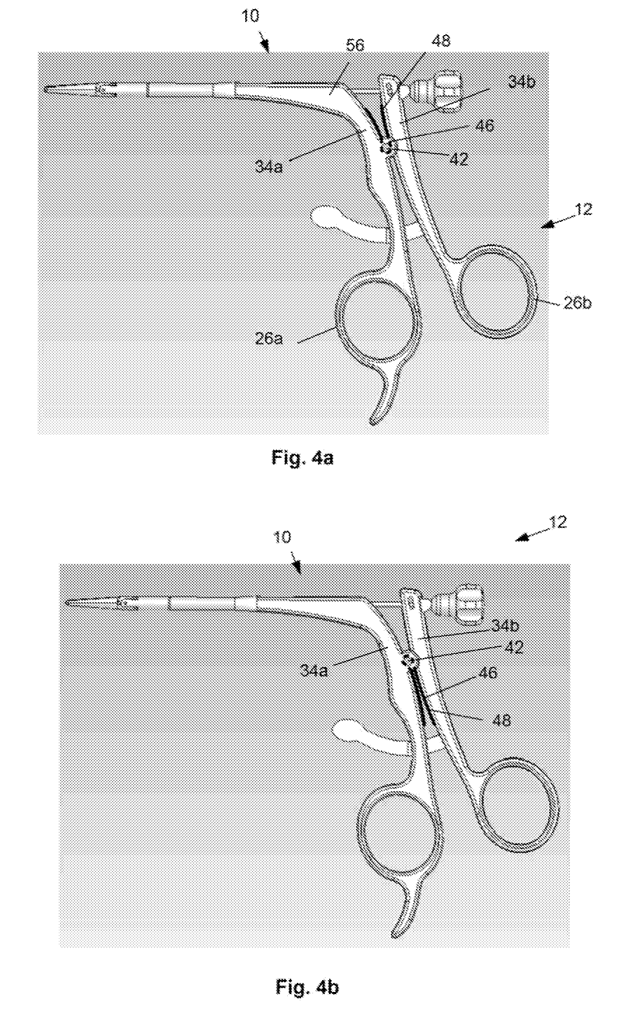

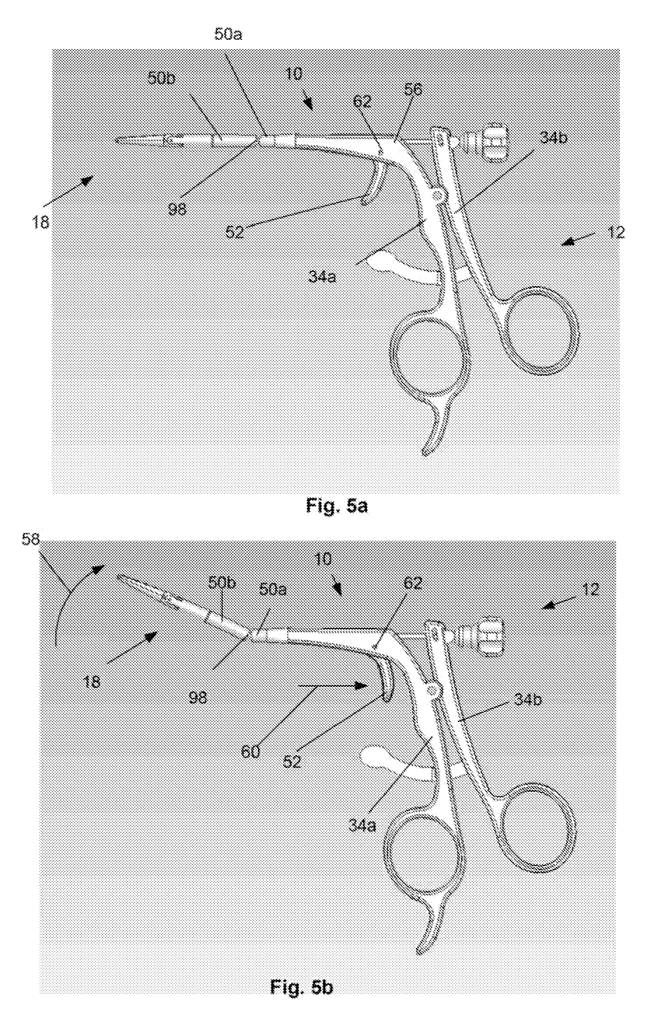

FIGS. 4a and 4b illustrate that the deployment tool 10 can have a return spring. The return spring can be a leaf or coil spring. The return spring can be configured to deploy a force or torque to the first handle 34a and/or second handle 34b so the first digit loop 26a rotates toward or away from the second digit loop 26b. The return spring can be configured to be attached to or integral with or around the handle hinge 42, first handle 34a, second handle 34b, or a combination thereof. As shown in FIG. 4a, the legs 48 of the return spring can point from the handle hinge 42 toward the breech 56. As shown in FIG. 4b, the legs 48 of the return spring 46 can point from the handle hinge 42 toward the digit loops.

The deployment tool 10 can have more than one return spring 46. For example, a first return spring can be biased against an oppositely configured second return spring (e.g., a combination of the return springs shown in FIGS. 4a and 4b). In such a variation of the deployment tool 10, the first handle 34a can have a resting angle with respect to the second handle 34b. When the first handle 34a is rotated with respect to the second handle 34b in a first direction away from the resting angle, the first return spring can deploy a resisting force/torque. When the first handle 34a is rotated with respect to the second handle 34b in a second direction away from the resting angle, the second return spring can deploy a resisting force/torque.

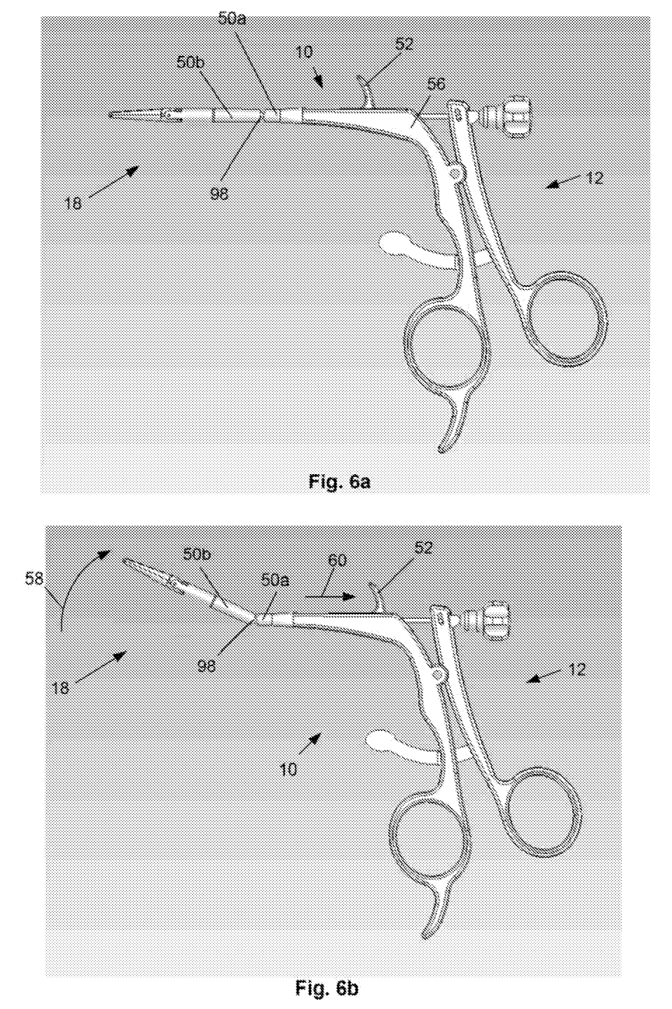

FIG. 5a illustrates that the deployment tool 10 can have an articulatable neck 16 and a neck controller, such as a trigger 52. The trigger 52 can be slidably and/or rotatably attached to the deployment tool 10, for example, with a trigger pin 62. The trigger 52 can extend from the breech 56 in the direction of the handles 34. The trigger 52 can extend coplanar or out of plane with the handles 34.

The neck 16 can have a neck first length 50a rotatably attached to a neck second length 50b, for example at a neck hinge 98.

Rotation of the trigger 52 can translate and/or rotate an internal articulation control rod (not shown) that can be configured to rotate the neck second length 50b. The articulation control rod can be a solid or hollow cylinder and/or a braided wire. The articulation control rod can be flexible and deformable and/or resilient.

FIG. 5b illustrates that depressing, as shown by arrow, the trigger 52 can result in rotation of the neck second length 50b and clamp 18, as shown by arrow. The depressing can be translation and/or rotation of the trigger 52. For example, the trigger 52 can be attached to a hinge, such as the trigger pin 62 as shown, and/or the trigger 52 and/or trigger pin 62 can be held in a straight or arcuate groove in the breech 56, allowing for translation and/or rotation. When the trigger 52 is fully depressed, a ratchet internal to the deployment tool 10 can catch and hold the trigger 52, articulation control rod, neck hinge 98, neck second length 50b, or a combination thereof in the articulated configuration.

FIG. 6a illustrates that the trigger 52 can extend away from the handles 34. FIG. 6b illustrates that the trigger 52 can be depressed, as shown by arrow 60. Depressing the trigger 52 can result in the rotation of the neck second length 50b and clamp 18, as shown by arrow 58.

The trigger 52 can be rotated about the longitudinal axis of the breech 56, or translated in a similar direction (e.g., nudged laterally). The ratchet can be released and/or the neck 16 can otherwise return to an unarticulated configuration, for example, when the trigger 52 is rotated about the longitudinal axis of the breech 56 or translated in a similar direction, and/or the trigger 52 is translated or rotated in the opposite direction as shown in FIGS. 5b and 6b.

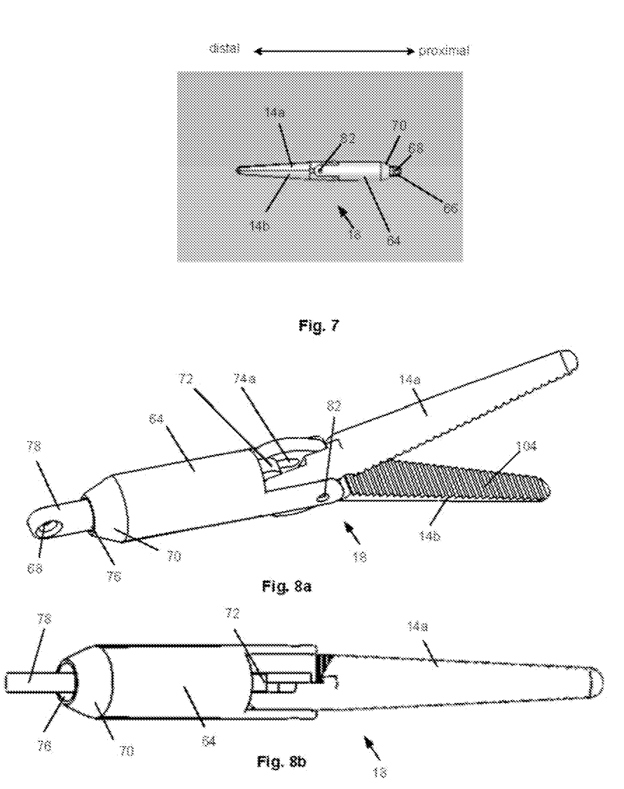

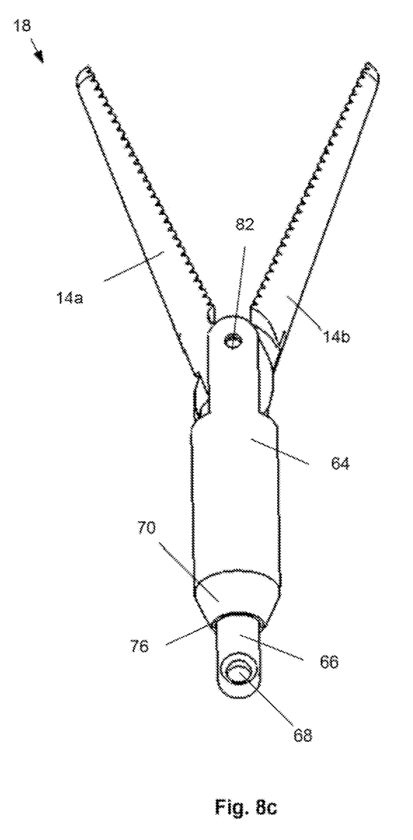

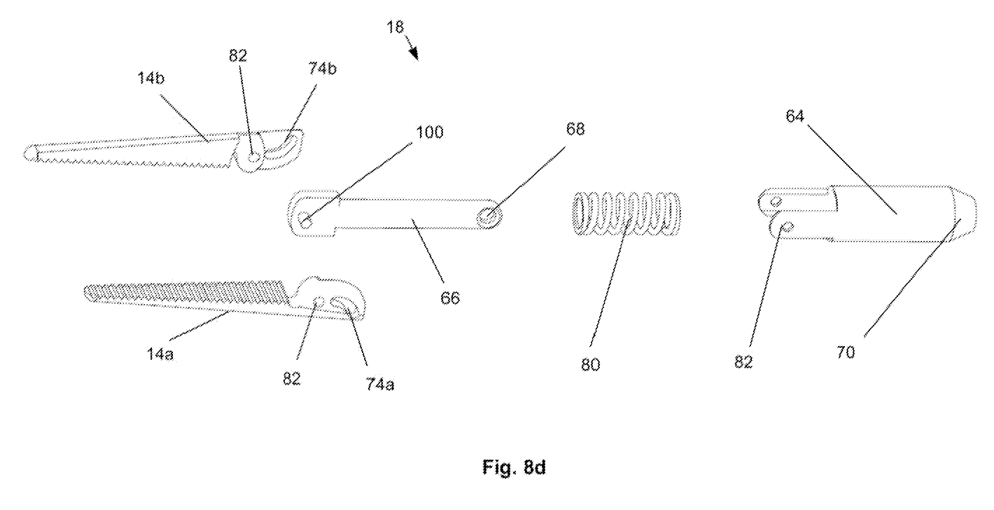

FIGS. 7, 8a, 8b, 8c, and 8d illustrate that the clamp 18 can have a clamp case 64. The clamp case 64 can be integral with, or fixedly or rotatably attached to the clamp hinge 82. The proximal end of the clamp case 64 can have a clamp abutment 70. The clamp abutment 70 can be configured to abut the distal end of the neck 16. For example, the clamp abutment 70 can have an inward or outward conical or cropped conical configuration.

A jaw activation rod 66 can extend through the clamp case 64. The jaw activation rod 66 can be slidably attached to the clamp case 64. The jaw activation rod proximal end 78 can extend out of the clamp case 64 at a clamp abutment port 76. The jaw activation rod distal end 72 can extend out of the clamp case 64 at a distal end of the clamp case 64.

The jaw activation rod proximal end 78 can have a jaw activation rod connection port 68 or other configuration, for example, for releasably attaching directly or indirectly to the deployment rod 20.

The first and second jaws 14a and 14b can each have jaw faces 104. The jaw faces 104 can be textured (e.g., knurled rough coated), and/or can be smooth (e.g., unfinished, polished, smooth coated), and/or can have attachment devices attached thereto (e.g., hook and loop configurations, brads and corresponding opposed ports). For example, a small minority portion at the distal end of the jaw face 104 can be smooth, and the remainder of the jaw face 104 can be textured.

The first jaw 14a can have a first jaw track 74a or slot. The second jaw 14b can have a second jaw track 74b or slot. The jaw tracks 74 or slots can be cam tracks or slots or be otherwise cam-like. The jaw tracks 74 can be at or near the proximal ends of the jaws 14. The jaw tracks 74 can have a straight or round (i.e., curved) configuration. For example, the jaw tracks 74 can have an arcuate or crescent configuration. The jaw tracks 74 can have constant or increasing or decreasing radii of curvature. The first jaw track 74a can curve or slope in the opposite direction oldie second jaw track 74b. For example, the first jaw track 74a can be concave up and the second jaw track 74b can be concave down.

The jaw activation rod proximal end 78 can have a first and/or second jaw control peg extending from one or both sides of the jaw activation rod proximal end 78. The control pegs can be cams. The control pegs can have circular or cam cross-sectional configurations. The control pegs can be configured to slidably attach in the adjacent jaw tracks 74. The jaws 14 can have pegs and/or the activation rod can have tracks.

FIG. 8d illustrates that the clamp hinge 82 can have components (e.g., ports and/or pins) on the first jaw 14a, the second jaw 14b and the clamp case 64. A separate clamp hinge pin (not shown) can be inserted through ports on the clamp case 64, first jaw 14a and second jaw 14b.

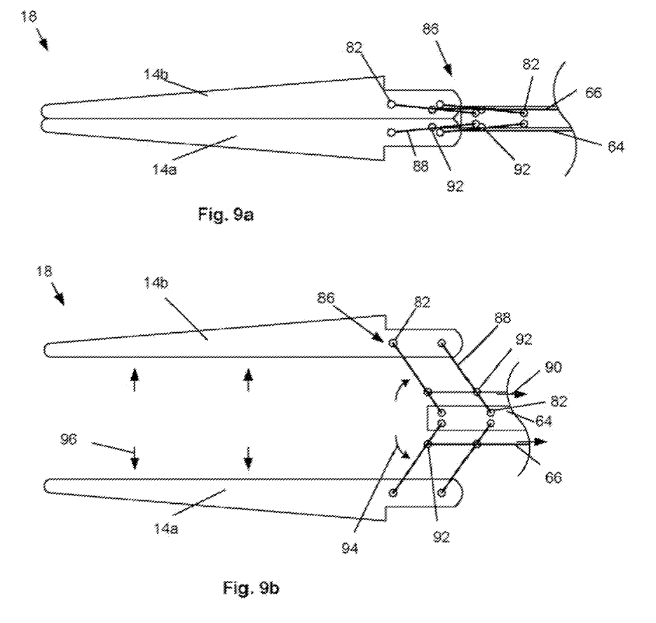

FIGS. 9a and 9b illustrate that a parallel jaw clip can have a four-bar linkage 86 to open and close the first and second jaws 14a and 14b in a parallel configuration. The clamp 18 can have pairs of rotatable rigid elements, for example bars 88 of the linkage. First ends of the bars can be affixed to simple clamp hinges 82, for example, two bars 88 can attach to the proximal end of each jaw. Second ends of the bars 88 can attach to clamp hinges 82 on clamp case 64. One or more jaw activation rods 66 can attach to the bars 88 at activation hinges 92. The bars 88 can be attached to the jaws 14 to open and close the jaws 14 while maintaining the jaws 14 in parallel or substantially parallel configurations with each other. The jaw activation rods 66 shown in FIGS. 9a and 9b can be separate or combine into a single rod proximal to the clamp 18.

FIG. 9a illustrates that the jaws 14 can be in a substantially closed configuration. FIG. 9b illustrates that a jaw activation rod pull force, as shown by arrows 90, can be applied separately and individually to one or concurrently and equally to both of the jaw activation rods 66. The jaw activation rods 66 can transmit the jaw activation rod pull force through the activation hinge 92, resulting in the four-bar linkage rotation, as shown by arrows 94. As one or both four-bar linkages 86 rotate, the jaws 14 can expand away H from each other, as shown by arrows 96, while maintaining a parallel configuration.

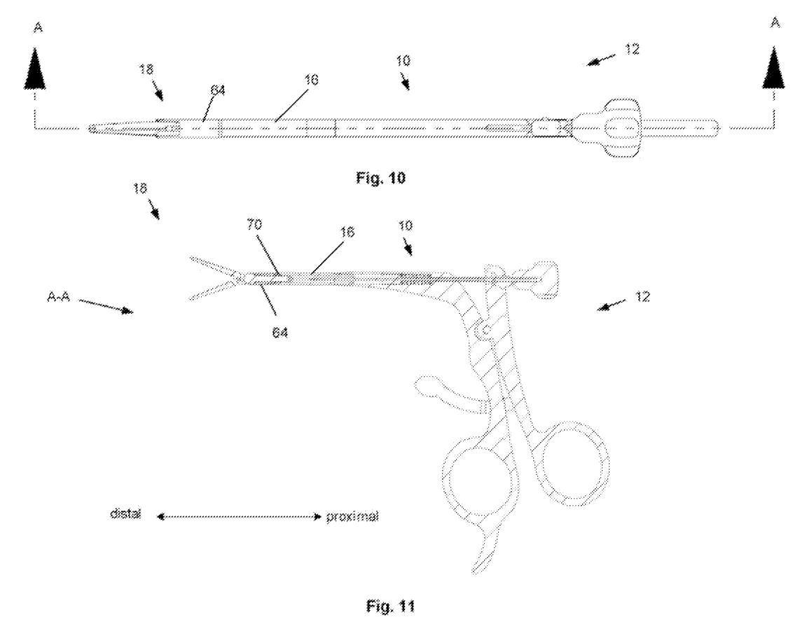

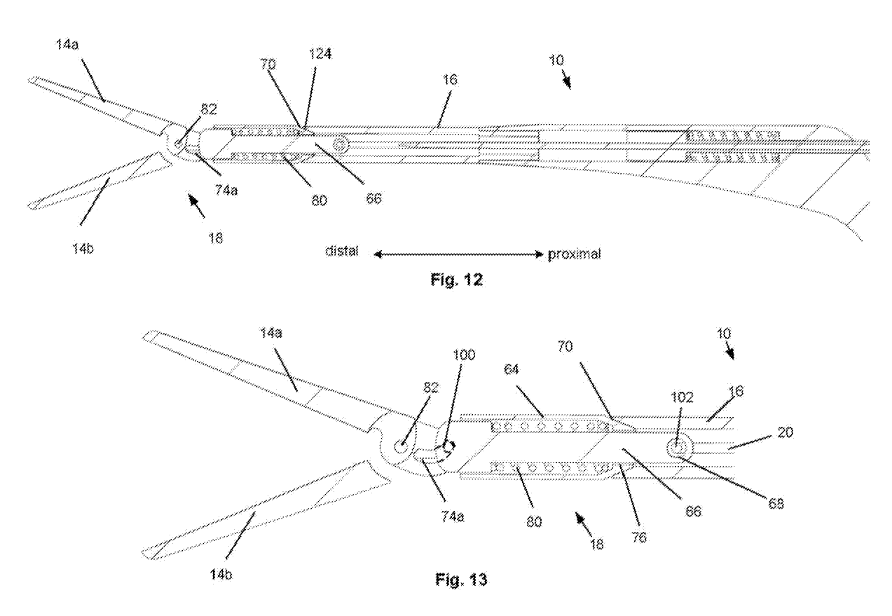

FIG. 10 illustrates a top view of a variation of the clamp deployment system 12 with the clamp 18 in an opened configuration. FIG. 11 illustrates cross-section A-A of FIG. 9. FIG. 12 illustrates a close-up of the distal end of cross-section A-A of the clamp deployment system 12 including the neck 16 and clamp 18. FIG. 13 illustrates an even closer view of the distal end of cross-section A-A of the clamp deployment system 12 than FIG. 12.

FIGS. 11, 12 and 13 illustrate that the clamp 18 can be removably attached to the deployment tool 10. The clamp 18 can have a jaw activation rod 66 that can be configured to removably attach to the deployment rod 20. The distal end of the deployment rod 20 can be attached to or integral with a deployment rod connector 102. The deployment rod connector 102 can be configured to be a hook or loop. The deployment rod connector 102 can be or have a snap, latch, brad, rivet, clamp, snare or combinations thereof.

The proximal end of the jaw activation rod 66 can be attached to or integral with a jaw activation rod connector. The jaw activation rod connector can be configured to releasably attach to the deployment rod connector 102. For example, the jaw activation rod connector can be a jaw activation rod connection port 68. The jaw activation rod connection port 68 can be configured to releasably attach to the hooked configuration of the deployment rod connector 102. The jaw activation rod connector can be a snap, latch, brad, rivet, clamp, snare or combinations thereof.

The jaw activation rod 66 can directly or indirectly control the angle of rotation of the first and/or second jaw 14a and/or 14b. The jaw activation rod 66 can be integral with or attached to a first control peg 100 (shown in FIG. 13) and a second control peg (not shown, but extending away from the jaw activation rod 66 in the opposite direction of the first control peg 100, and sharing a longitudinal axis with the first control peg 100). The first control pen 100 can slidably attach to a first jaw track 74a. The first jaw track 74a can be in or on the first jaw 14a, for example at the proximal end of the first jaw 14a. The second control peg can slidably attach to a second control track. The second control track can be in or on the second jaw 14b, for example at the proximal end of the second jaw 14b.

The clamp case 64 can be slidably attached to the jaw activation rod 66. In a first configuration, a clamp spring 80 can be in compression between the clamp case 64 and the jaw activation rod 66. For example, a first end of the clamp spring 80 can press against the clamp case 64 and a second end of the clamp spring 80 can press against the jaw activation rod 66. The clamp 18 can be in an open configuration in the first configuration with the clamp spring 80 in compression.

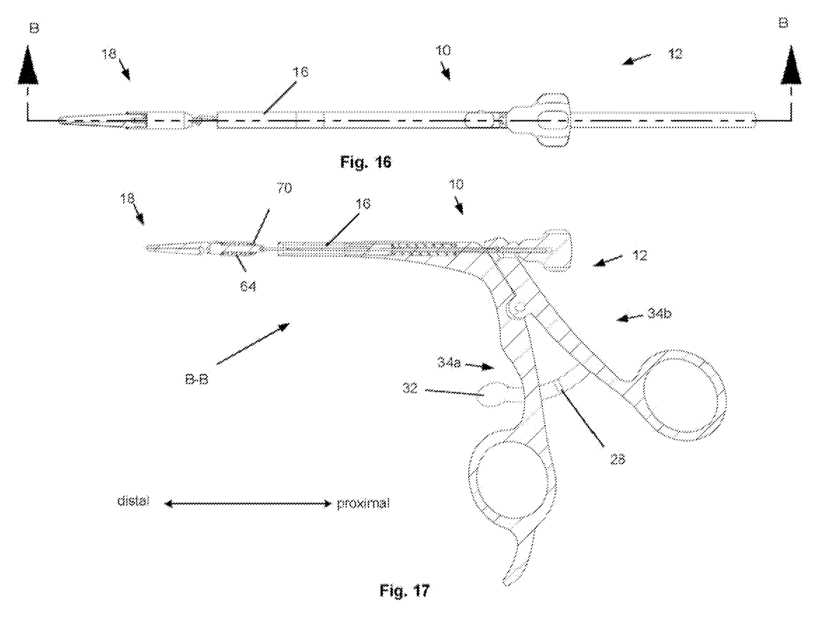

In the first configuration, for example with the clamp 18 in an open configuration, the deployment rod 20 can apply a proximal force to the jaw activation rod 66. The clamp case 64 can have a clamp abutment 70 at the proximal end of the clamp case 64. The clamp abutment 70 can be pressed against a neck seat 124 at the distal end of the neck 16. The jaw activation rod 66 can deliver the proximal force to the clamp spring 80, moving the jaw activation rod 66 proximally with respect to the clamp case 64. The clamp case 64 can have a stationary connection to the jaws 14 at the clamp hinge 82. Thus, the jaw activation rod 66 can be in a proximally translated configuration with respect to the jaws 14 when in a relatively relaxed configuration (as shown in FIGS. 16-18), for example, opening the jaws 14 by sliding the control pegs to the proximal ends of the jaw tracks 74.

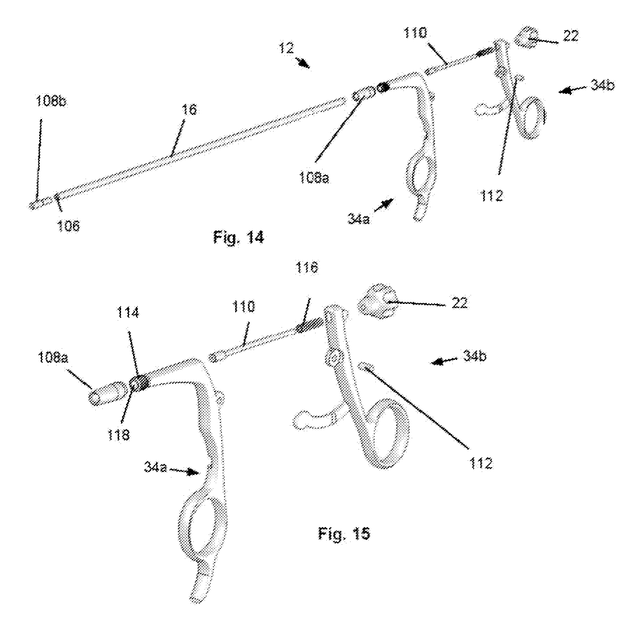

FIG. 14 illustrates that the clamp deployment system 12 can have a neck 16 separably attached to the first handle 34a. The neck 16 can be replaced with a neck 16 of a different length and/or diameter for different uses. For example, a longer neck 16 can be used to access deeper target sites. The neck 16 can have a hollow neck barrel 106. The distal end of the neck 16 can attach or fix to a distal barrel insert 108b. The distal barrel insert 108b can be press fitted, glued or otherwise attached to the radial inside of the neck barrel 106 and/or the radial outside of the neck 16.

FIGS. 14 and 15 illustrate that the first handle 34a can be rotatably and separably attached to the second handle 34b with a handle axle 112. The first handle 34a can have a handle connector 114 at the distal end of the first handle 34a. The handle connector 114 can have radially internal and/or radially external threads. The handle connector 114 can removably or permanently attach to the proximal barrel insert 108a and/or directly to the proximal end of the neck 16. The proximal and/or distal ends of the proximal barrel insert 108a can have radially internal and/or radially external threads. The proximal barrel insert 108a can removably or permanently attach to the first handle 34a (e.g., at the handle connector) and/or to the proximal end of the neck 16. The proximal barrel insert 108a can be hollow.

The first handle 34a can have a hollow first handle 34a barrel 118 through which a puller 110 can slide. The puller 110 can be attached to the second handle 34b.

A puller spring 116 can be slidably fined over the puller 110 between the first handle 34a and the second handle 34b. The puller spring 116 can be compressed between the first handle 34a and the second handle 34b. The puller spring 116 can force the puller 110 and/or deployment rod 20 proximally. When second handle 34b is rotated with respect to the first handle 34a during use to extend the deployment rod 20 and/or puller 110 distally, the rotation can be opposed by the puller spring 116.

The deployment rod anchor 22 can be attached to the puller 110 and/or the deployment rod 20. The deployment rod 20 can be attached to the puller 110. For example, the proximal end of the deployment rod 20 can be attached to the distal end of the puller 110 and/or the deployment rod 20 can slidably pass through a channel in the puller 110.

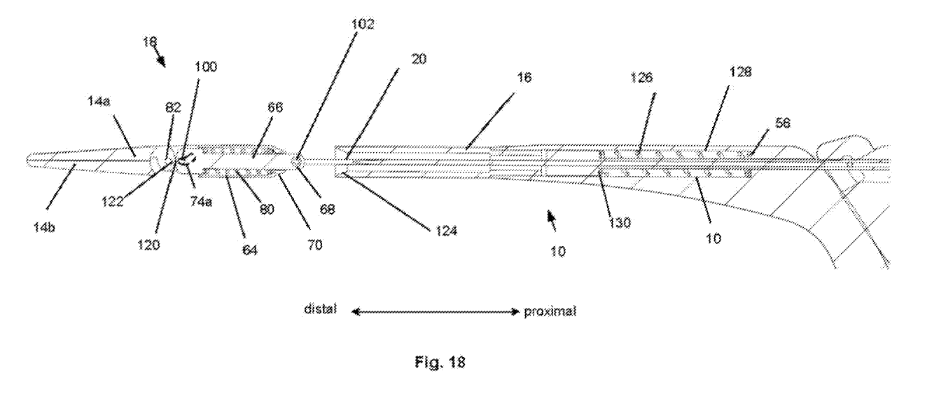

FIG. 16 illustrates a top view of a variation of the clamp deployment system 12 with the clamp 18 in a closed configuration. FIG. 17 illustrates cross-section B-B of FIG. 16. FIG. 18 illustrates a close-up of the distal end of cross-section B-B of the clamp deployment system 12 including the neck 16 and clamp 18.

FIGS. 17 and 18 illustrate that the clamp 18 can be in a closed configuration extended away (e.g., distally) from the neck 16. The first handle 34a and second handle 34b can be rotated away from each other, for example so that the first handle 34a is positioned between the marker 28 and the stop 32. The second handle 34b can be fixed to the deployment rod 20. The deployment rod 20 can translate in the distal direction, reducing the force exerted between the clamp abutment 70 and the neck seat 124, and (e.g., after the marker 28 passes the first handle 34a) extending the clamp 18 distally with respect to the neck 16.

The jaw activation rod 66 can translate distally with respect to the remainder of the clamp 18. For example, an activation rod abutment 120 at the distal end of the jaw activation rod 66 can interference fit against a jaw abutment 122 of the first and/or second jaws 14a and/or 14b proximal to the clamp hinge 82.

As the jaw activation rod 66 translates distally with respect to the remainder of the clamp 18, the jaw tracks 74 can apply force to the respective control pegs, causing the jaws to rotate. When the jaw activation rod 66 is at the most distal position, the clamp 18 can have a closed configuration and the faces of the opposing jaws can be substantially in contact with each other.

The neck 16 can have a deployment spring 126 in a deployment cylinder 128. The deployment spring 126 can be compressed between a deployment piston 130 and a breech 56. The breech 56 can be a static portion of the deployment tool 10. The deployment spring 126 can be radially internal or external to the deployment cylinder 128. For example, as shown by the return spring 46 in FIGS. 4a and 4b, the first handle 34a can be H sprang toward and/or away from the second handle 34b with a torsion spring installed at the handle hinge 42 (e.g., the rotational axis of the spring can be substantially identical to the rotational axis of the handle hinge 42), and/or can be sprung toward or away by a coil spring installed between the first and second handle 34b. The deployment piston 130 can be slidably attached to the deployment cylinder 128. The deployment piston 130 can be fixedly attached to, or integral with, the deployment rod 20. When there are no external forces on the handles 34 (e.g., when the user releases or relaxes his or her grip on the handles 34), the deployment spring 126 can push the deployment, piston 130 away from the breech 56.

The deployment spring 126 can be proximal to the deployment piston 130 to bias the clamp 18 to an extended and closed or non-extended and closed configuration when there are no external forces on the handles 34. The temporary interference force from the marker 28 against the first handle 34a can be a greater force than the force from the deployment spring 126 in order to bias the clamp 18 to a non-extended and closed configuration. Alternately, the spring force can be greater than the temporary interference force from the marker 28 against the first handle 34a to bias the clamp 18 to an extended and closed configuration.

The deployment spring 126 can be distal to the deployment piston 130 to bias the clamp 18 to a non-extended closed or opened, or the deployment to prevent the clamp 18 configuration when there are no external forces on the handles 34. The temporary interference force from the marker 28 against the first handle 34a can be a greater force than the force from the deployment spring 126 to bias the clamp 18 to a closed configuration. Alternately, the spring force can be greater than the temporary interference force from the marker 28 against the first handle 34a to bias the clamp 18 to an opened configuration.

With the clamp 18 extended away from the deployment tool 10, the clamp abutment 70 can be out of contact with the neck seat 124. When no substantial force on the clamp case 64 is exerted from outside of the clamp 18 (e.g., from the neck seat 124), the clamp spring 80 can force the rod distally with respect to the remainder of the clamp 18.

The second handle 34b and first handle 34a can be rotated together and held in compression (or relaxed, depending on the configuration of the handles 34 and springs, as understood by one having ordinary skill in the art with this disclosure), for example to maintain the clamp 18 in an opened configuration.

The first and second handle 34b can be released from compression (or relaxed, depending on the configuration of the handles 34 and springs, as understood by one having ordinary skill in the art with this disclosure) and rotated apart compared to when the clamp 18 is in an opened configuration, for example to maintain the clamp 18 in a closed configuration not extended from the neck 16. In the configuration with the clamp 18 in a closed configuration not extended from the neck 16, the marker 28 can abut and temporary interference fit against the first handle 34a.

The first and second handle 34b can be rotated apart in tension, for example to maintain the clamp 18 in a closed configuration extended from the neck 16. In the configuration with the clamp 18 in a closed configuration extended from the neck 16, the stop 32 can abut and interference fit against the first handle 34a.

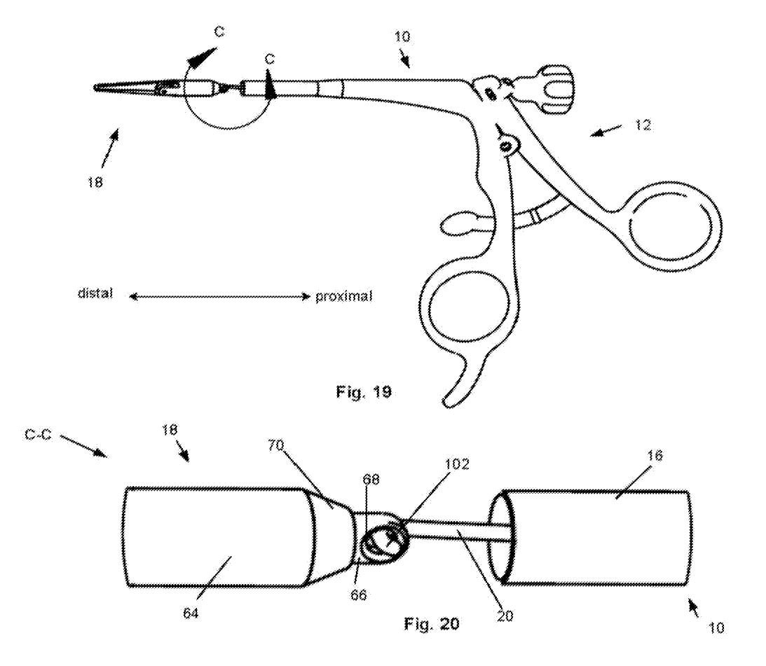

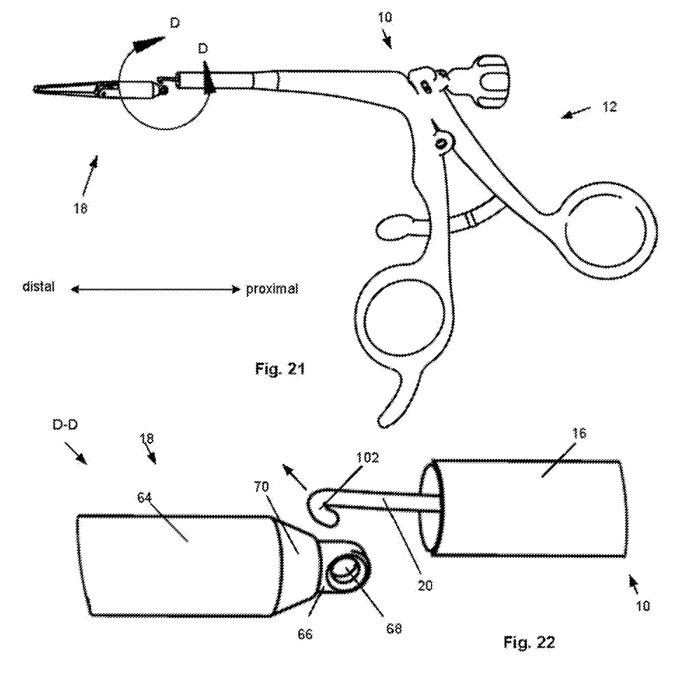

FIGS. 19 and 20 illustrate that the deployment rod connector 102 can be hooked into, or otherwise directionally attached to the jaw activation rod connection port 68. FIGS. 21 and 22 illustrate that the deployment tool 10 can be translated in a combined distal and lateral direction with respect to the clamp 18. The clamp 18 can be in a substantially fixed location when in a closed configuration against a target vessel 144. The deployment rod connector 102 can detach from the jaw activation rod connection port 68.

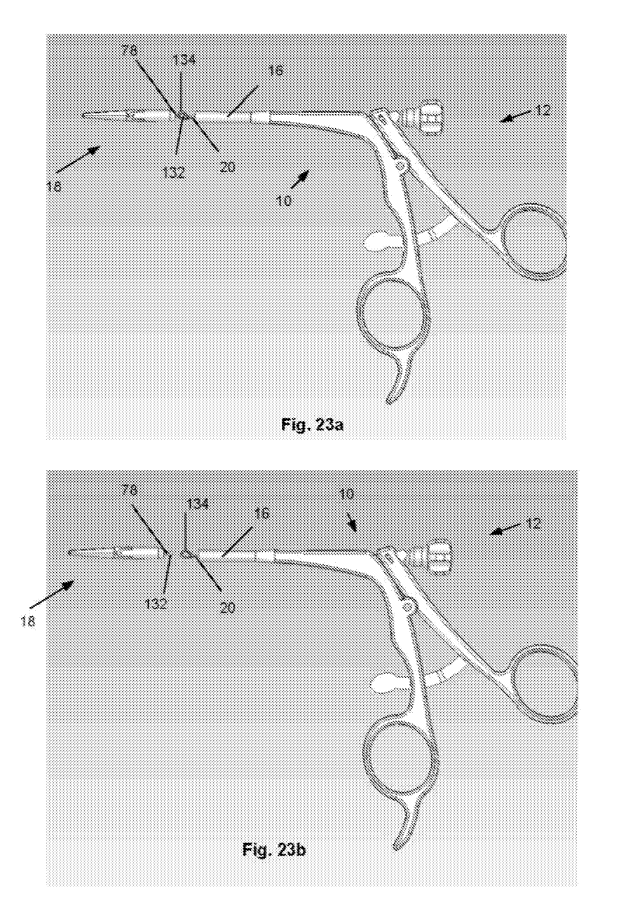

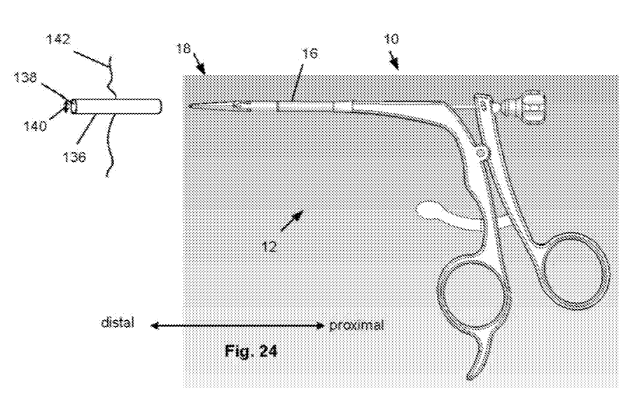

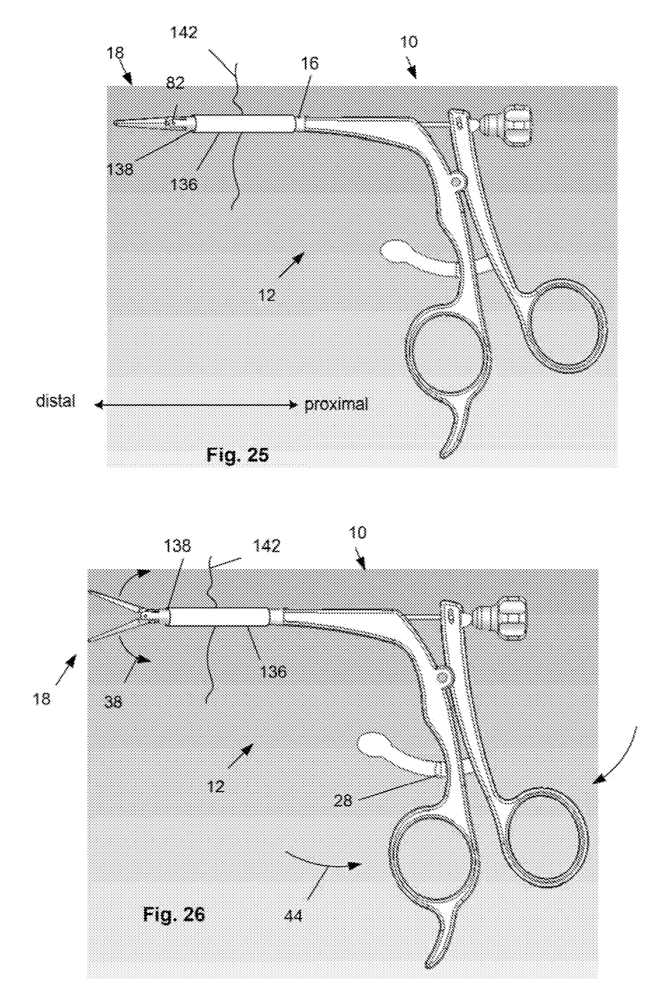

FIG. 23a illustrates that the jaw activation rod connector can be a ball 132, for example positioned at or near the terminal end of the jaw activation rod proximal end 78. The deployment rod connector 102 can be a snare 134. The snare 134 can be positioned at the terminal end of the deployment rod 20 nearest the clamp 18. The ball 132 can be configured to releasably attach to the snare 134. The snare 134 can be configured to releasably attach to the snare 134.

FIG. 23b illustrates that the champ can be detached from the deployment tool 10 by detaching the ball 132 from the snare 134. In one variation, the snare 134 can be twisted, translated, or otherwise shifted to release from the ball 132, and/or the ball 132 can be twisted, translated, or otherwise shifted to release from the snare 134. In another variation, the clamp 18 can be secured over a target site such as a target lumen 146 or other target tissue. The snare 134 can then be pulled off the ball 132 by translating the deployment tool 10 away from the clamp 18, while the clamp 18 maintains an interference or friction fit against the target site.

The deployment tool 10 can be re-attached to the clamp 18, for example by re-inserting the deployment rod connector 102 into the jaw activation rod connection port 68. The clamp 18 can then be opened, repositioned on the same target vessel or a different target vessel, and reclosed, or opened, repositioned away from tissue, closed, and removed from the patient.

The clamp deployment system 12 can be configured so the clamp 18 is substantially aligned along a longitudinal axis that passes through the distal end of the neck 16 (i.e., so the clamp 18 can not swivel or rotate with respect to the distal end of the neck 16). Alternately, the clamp deployment system 12 can be configured so the clamp 18 can swivel or rotate with respect to to distal end of the neck 16.

The neck 16 can be rigid and not rotated or bend, or be flexible and capable of rotating or bending. The neck 16 can be rigid and made of multiple parts so that a controllable means of articulation of the clamp 18 away from the longitudinal axis is possible.

Any or all elements of the clamp deployment system 12 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET/polyester (e.g., DACRON® from E. I. Du Pont de Nemours and Company, Wilmington, Del.), polypropylene, (PET), polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, it biomaterial (e.g., cadaver tissue, collagen, allograft, autograft, xenograft) any of the other materials listed herein or combinations thereof. Examples of radiopaque materials are barium sulfate, zinc oxide, titanium, stainless steel, nickel-titanium alloys, tantalum and gold. Any or all elements of H the clamp deployment system 12 or combination thereof may be either disposable or sterilizable for reuse.

### METHODS OF USE

FIG. 24 illustrates that a cannula 136, such as a trocar, can be deployed across a tissue surface 142. The tissue surface 142 can be skin, an organ membrane or surface, a body cavity membrane (e.g., peritoneal membrane, pericardial membrane), or combinations thereof (e.g., the tissue surface 142 can be representative of skin and one or more membranes).

The cannula 136 can have an internal cannula channel 138. The cannula channel 138 can have a cannula inner diameter 140. The cannula inner diameter 140 can be from about 2.0 mm (0.079 in.) to about 5 mm (0.20 in.), for example about 4 mm (0.16 in.).

The clamp deployment system 12 can include and/or be separate from the cannula 136. (The clamp deployment system 12 is described separately from the cannula 136 hereafter for clarity.) The clamp deployment system 12 can be positioned adjacent to the cannula 136. The distal end of the clamp 18 can be aligned with an end of the cannula channel 138.

FIG. 24 illustrates that the clamp deployment system 12 can be translated into and through the cannula 136, for example, while the clamp 18 is in a closed configuration. The clamp 18 can be positioned substantially distal to the cannula 136. The clamp hinge 82 can be positioned substantially distal to the cannula 136.

FIG. 26 illustrates that the first handle 34a can be rotated, as shown by arrows, toward the second handle 34b. The clamp 18 can open, as shown by arrows. The handles 34 can be rotated to a relative angle between the handles 34 so the marker 28 is positioned outside of the angle created by the first and second handle 34bs. The first handle 34a can abut the second handle 34b.

FIG. 27 illustrates that the open clamp 18 can be positioned around a target vessel 144. The target vessel 144 can have a lumen 146 which is desired to be partially or completely occluded. The target vessel 144 can be a blood vessel (e.g., hepatic, portal, coronary, splenic, pulmonary, or renal veins or arteries), ureter, urethra, intestine, esophagus, hepatic or bile duct, pancreatic duct, tear duct, fallopian tube, vas deferens, or other biological lumen. Even though shown as a target vessel 144, the clamp 18 can be placed on a membrane, for example a folded portion of skin, peritoneal membrane or pulmonary membrane.

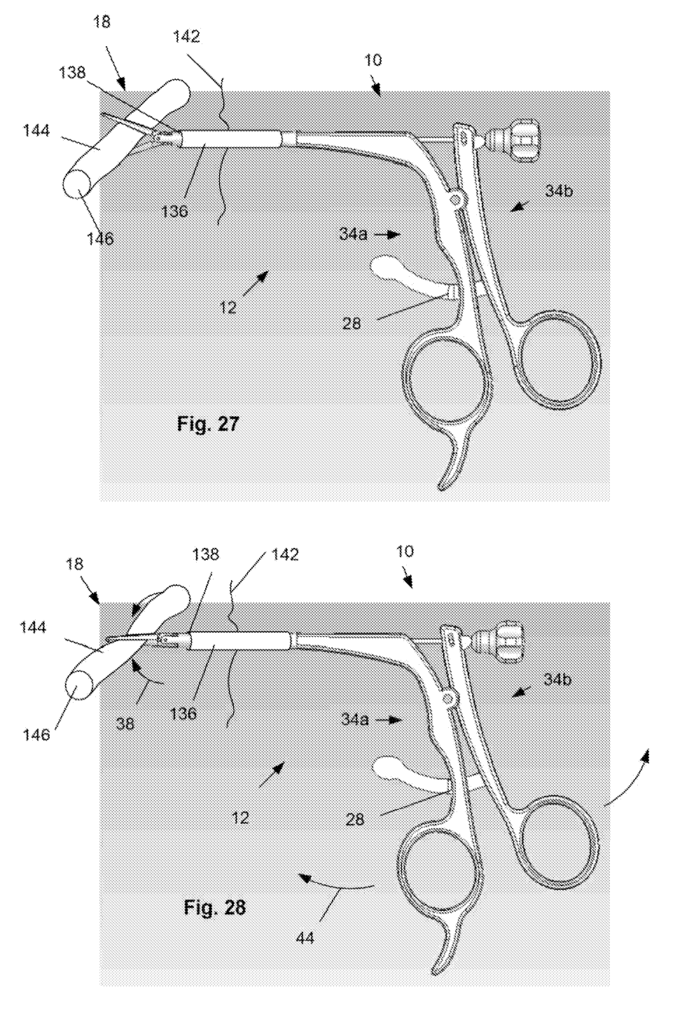

FIG. 28 illustrates that the first handle 34a can be rotated away from the second handle 34b, as shown by arrows 44. The marker 28 can abut and temporarily interference fit against the first handle 34a. The clamp 18 can partially or completely close (e.g., jaws can rotate, as shown by arrows) on the vessel 144. The lumen 146 can be partially or completely occluded.

FIG. 29 illustrates that the first handle 34a can be rotated away from the second handle 34b, as shown by arrows, forcing the marker 28 between the first handle 34a and the second handle 34b. The clamp 18 can extend away from the neck 16, as shown by arrow 36. The clamp 18 can remain in a substantially closed configuration during extension away from the neck 16. The user can rotate the handles 34 to a desired angle where the first handle 34a is positioned between the marker 28 and the stop 32. For example, the handles 34 can be rotated so that the stop 32 abuts the first handle 34a. In the configuration where the clamp 18 is extended from the neck 16, the deployment tool 10 can be retracted proximally and/or the vessel 144 can be forced distally. For example, the vessel can be manipulated by the clamp 18.

The clamp 18 can then be detached from the deployment, rod 20, as shown in FIGS. 21 and 22, or 23b, and described above.

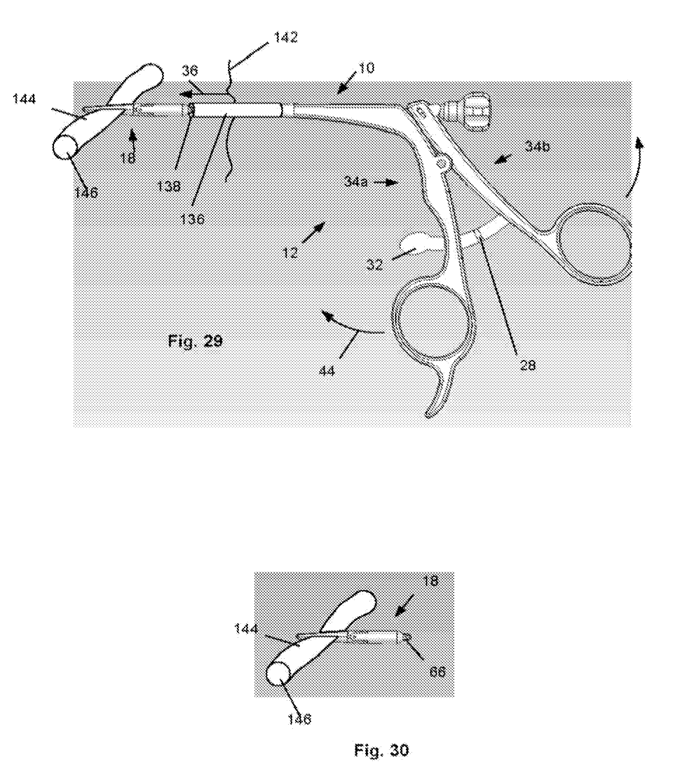

FIG. 30 illustrates that the clamp 18 can remain clamped on the vessel 144 and the deployment tool 10 can be removed from the patient. Alternately, the deployment tool 10 can be in patient, but not in the vicinity of the vessel 144 (e.g., if the doctor wants to check the clamp 18 without the deployment tool 10 nearby). The cannula 136 can be removed from the patient and the wound from the cannula 136 can be closed. The clamp 18 can remain in the patient temporarily or permanently.

The deployment tool 10 can be re-attached to the clamp 18 (e.g., by reversing the steps shown in FIG. 19-22, or 23b, and described above). The clamp 18 can then be re-opened and re-positioned, and re-closed on the same or a different vessel; or re-opened, moved away from the vessel 144, closed, and withdrawn through the cannula 136 and removed from the patient.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

1. A biological lumen clamp deployment system comprising:
a deployment tool comprising a neck,
a clamp releasably attached to the deployment tool, wherein the clamp has an open configuration and a closed configuration, and wherein the clamp has a substantially circular cross-section in the closed configuration, and
wherein the deployment tool is configured to re-attach to the clamp after releasing the clamp.

2. The system of claim 1, wherein the deployment tool and the clamp are configured to open the clamp when the clamp is pulled toward the neck, and wherein the deployment tool and the clamp are configured to close clamp when the clamp is pushed away from the neck.

3. The system of claim 1, wherein the clamp comprises a first jaw, a second jaw, and a hinge, and wherein the first jaw is rotatably attached to the second jaw at the hinge.

4. The system of claim 1, further comprising a cannula having a channel with a channel inner diameter, wherein the clamp and the neck are configured to be slidably inserted through the channel.

5. The method of claim 4 further comprising:
wherein the channel has an inner diameter less than about 5 mm.

6. The system of claim, wherein the channel inner diameter is less than about 5 mm.

7. The system of claim 1, wherein the clamp is controllably openable and closeable by the deployment tool.

8. The system of claim 1, wherein the neck is flexible.

9. The system of claim 1, wherein the neck is rigid.

10. A biological lumen clamp deployment system comprising:
a deployment tool comprising a neck,
a clamp releasably attached to the deployment tool, wherein the clamp has an opened configuration and a closed configuration,
wherein the deployment tool is configured to extend the clamp away from the neck while the clamp is in the closed configuration and before the deployment tool releases from the clamp.

11. The system of claim 10, wherein the deployment tool is configured to deliver feedback when the clamp is closed and immediately before the clamp is extended away from the neck.

12. The system of claim 10, wherein the deployment tool and the clamp are configured to open the clamp when the clamp is compressed against the neck, and wherein the deployment tool and the clamp are configured to close clamp when the clamp is not compressed into the neck.

13. The system of claim 10, wherein the clamp comprises a first jaw, a second jaw, and a hinge, and wherein the first jaw is rotatably attached to the second jaw at the hinge.

14. The system of claim 10, wherein the clamp is controllably openable and closeable by the deployment tool.

15. The system of claim 10, further comprising a cannula having a channel with a channel inner diameter, wherein the clamp and the neck are configured to be slidably inserted through the channel, wherein the channel inner diameter is less than about 5 mm.

16. The system of claim 10, wherein the neck is flexible.

17. The system of claim 10, wherein the neck is rigid.

18. The method of deploying a biological lumen clamp into a subject, wherein the lumen clamp comprises a radially expandable and contractable clamp, a neck, and a control element, and wherein the clamp comprises a first jaw and a second jaw, the method comprising:
inserting a cannula percutaneously in the subject, wherein the cannula has a channel, and wherein a first end of the cannula is outside the subject, and wherein a second end of the cannula is inside the subject,
inserting the clamp and the neck through the channel, wherein at least part of the control element remains outside the subject,
positioning the clamp outside of the cannula and inside the subject,
radially expanding the clamp, comprising reducing a compression of the clamp against the neck,
moving the clamp around a lumen,
radially contracting the clamp, and
detaching the clamp from the control element.

19. The method of claim 18, further comprising:
re-attaching the clamp to the control element,
radially expanding the clamp after re-attaching the clamp to the control element,
repositioning the clamp around the lumen,
radially contracting the clamp and
detaching the clamp from the control element.

20. The method of claim 18, further comprising:
re-attaching the clamp to the control element,
radially expanding the clamp after re-attaching the clamp to the control element,
radially contracting the clamp after re-attaching the clamp to the control element, and
withdrawing the clamp through the channel.

21. The method of claim 20, wherein radially expanding comprises moving the clamp toward the neck, and wherein radially contracting comprises moving the clamp away from the neck.

22. The method of claim 20, wherein radially expanding comprises abutting the clamp into the neck.
